(19) United States
(12) Patent Application Publication
(10) Pub. No.: US 2023/0031799 A1
(43) Pub. Date: Feb. 2, 2023

(54) NERVE INJURY PROTECTION DEVICE

(71) Applicant: LANKENAU INSTITUTE FOR MEDICAL RESEARCH, Wynnewood, PA (US)

(72) Inventors: Boris Aronzon, King of Prussia, PA (US); Michael Furr, Kennett Square, PA (US); Christopher Scholl, West Chester, PA (US); Aidan Hyde, Collingswood, NJ (US); Ray Czerwinski, Downingtown, PA (US)

(21) Appl. No.: 17/964,415

(22) Filed: Oct. 12, 2022

Related U.S. Application Data
(63) Continuation of application No. PCT/US21/29587, filed on Apr. 28, 2021.
(60) Provisional application No. 63/016,586, filed on Apr. 28, 2020.

(51) Int. Cl. A61B 46/27; A61B 46/00

(52) U.S. Cl. CPC A61B 46/27; A61B 46/40; A61B 2046/205

(57) ABSTRACT

A protective device that protects against nerve compression and injury during medical procedures. The device includes an elongated body having a base, and a retention flap and a closure flap joined to the base. The device further includes a closure operable to join the closure flap to the retention flap. The closure may be provided as adhesive field, magnets, a living hinge, or matable structures, such as teeth/sockets, snaps, etc., or tape, strings, sutures or other external elements may be used for joining the flaps. The body may be pretensioned to remain open, or to urge the body to assume a closed configuration. The body may be formed of foam or other flexible/compressible material so as to readily bend and conform to the body and to act to enclose and organize/confine wires/lines used in a medical procedure, and to act as a cushion between such wires/lines and the body.

### CROSS-REFERENCE TO RELATED APPLICATION

This application is a continuation of PCT International Application No. PCT/US21/29587, filed Apr. 28, 2021, which claims the benefit of priority, under 35 U.S.C. § 119(e), of U.S. Provisional Patent Application No. 63/016,586, filed Apr. 28, 2020, the entire disclosure of which is hereby incorporated herein by reference.

### FIELD OF THE INVENTION

The present invention relates generally to devices for use in the operating theater or otherwise in connection with medical treatment or care, and more particularly, to a protective device for use to protect the arm, hand and/or other limb/body regions of a patient from nerve injuries caused by contact of medical procedure-related cables, tubes and like with the patient's skin, which creates pressure points during the medical treatment, while also protecting the cables, tubes and the like from damage, crushing and/or pinching that would tend to interfere with their operation, fluid administration, or other intended purposes.

### DISCUSSION OF RELATED ART

Various surgeries, medical treatments, and other aspects of medical and health care (collectively referred to herein as “procedures”) involve the use of intravenous (IV) tubing, which has associated Luer locks, Y-piece connections and/or other fittings, and/or other tubing, and/or cables, leads, wires and/or other elongated members, such as those used in connection with pulse oximetry and/or blood pressure monitoring apparatuses (collectively referred to herein as “medical lines”). Often, as part of proper medical procedure and/or convenience, these medical lines are positioned to extend along, rest upon and/or abut the patient's arm and/or hand regions.

Additionally, many of these procedures, and particularly surgeries, involve a need to place and retain the patient's arms by the patient's side during the procedure. This is particularly true of many surgeries, including, for example, robotic abdominal surgeries or pelvic laparoscopy, laparotomy, cystoscopy, colorectal, and vulvovaginal surgery and other surgeries and procedures in which patients are placed in the lithotomy position or any position that requires robotic or laparoscopy access or when patients' arms are tucked (e.g., in head and neck surgery). It is typical in these procedures to restrain the patient's arms during the procedure, e.g., by wrapping the patient's arms relatively tightly in sheets that are then tucked tightly against the patient's trunk/tors, and/or using surgical arm protectors that are wrapped relatively tightly around the patient's arms and then secured to an arm board or accessory rail of a surgical table, or otherwise. While this is effective in restraining the patient's arms and in avoiding instability of the patient and/or certain types of harm to the patient and/or interference with the procedure, it often results in creation of pressure points that result in ulnar, medial or radial nerve compression and/or other injury to the nerves in the patient's arms as a result of forcible impingement of the medical lines upon the skin and nerve tissue of the patient, often for protracted periods of time during the procedure.

With the exponential growth of robotic surgery, and the continued prevalence of laparoscopic surgery, increasing numbers of surgeries are performed in which patients' arms needs to be restrained during the surgery. For example, it is estimated that approximately 400,000 robotic surgeries were performed across all types of surgery in the United States in 2012, and that the rate of robotic surgeries is increasing by 25 percent annually. Accordingly, nerve injuries of this type are likely to increase.

Nerve injuries of this type are undesirable for many reasons, including neuropathy and/or other impacts upon the patient, the possible need for corrective surgery and/or procedures to aid in recovery from the injury, or extensive and costly rehabilitation treatment, dissatisfaction of the patient, and potential medical malpractice claims, all of which are undesirable.

Additionally, these medical lines serve important purposes with respect to the medical procedure, and thus it is desirable that be permitted to operate properly. The tight wrapping of these medical lines along with the patient's arms exposes these medical lines to a risk of crushing, pinching and/or kinking (collectively referred to herein as “damage”) that can interfere with their operation as intended. This is undesirable for many reasons, including an adverse impact to the patient, repositioning of the patient during surgery, additional operating room time and material cost, and resulting medical malpractice claims.

Accordingly, there is a need for a device that is compatible with conventional procedures and techniques for protection and restraint of the patient's arms during procedures and that protects the patient's arms from nerve compression and injury resulting from compression of nerves by medical lines associated with such procedures, which also protecting the medical lines themselves from damage that would interfere with operation for their intended purposes.

### SUMMARY

The present invention provides a protective device that protects the patient's arms from nerve compression and injury resulting from compression of nerves by medical lines associated with such procedures, which also protecting the medical lines themselves from damage that would interfere with operation for their intended purposes. The protective device is compatible with conventional procedures and techniques for protection and restraint of the patient's arms during procedures. In one embodiment, a protective device in accordance with the present invention comprises a body comprising an elongated base and a retention flap, joined to the base, that extends longitudinally relative to the base. The device further includes a closure flap joined to the base. The retention flap extends longitudinally relative to the base opposite the retention flap. The device further includes a closure supported on the body and operable to join the closure flap to the retention flap.

### BRIEF DESCRIPTION OF THE FIGURES

An understanding of the following description will be facilitated by reference to the attached drawings, in which:

FIGS. 1A and 1B are perspective views of a nerve injury protection device in accordance with an exemplary embodiment of the present invention;

FIG. 2 is a perspective view of an alternative embodiment of a nerve injury protection device in accordance with the present invention;

FIG. 3 is a perspective view of a nerve injury protection device in accordance with another alternative embodiment of the present invention;

FIGS. 4A and 4B are perspective views of a nerve injury protection device in accordance with yet another alternative embodiment of the present invention;

FIGS. 5A and 5B are perspective views of a nerve injury protection device in accordance with yet another alternative embodiment of the present invention;

FIGS. 6A and 6B are perspective views of yet another nerve injury protection device in accordance with yet another alternative embodiment of the present invention;

FIGS. 7A and 7B is a perspective view of yet another nerve injury protection device in accordance with yet another alternative embodiment of the present invention;

FIG. 8 is a perspective view of an exemplary dispenser device for a nerve injury protection device in accordance with the present invention; and

FIG. 9 is a top view of an exemplary nerve injury protection device in accordance with the present invention, shown in use with a portion of the device removed for illustrative clarity.

### DETAILED DESCRIPTION

The present invention provides a nerve injury protection device (also referred to herein as a protective device) that is configured to protect the patient's arms (or other limbs or body parts) from nerve compression and injury resulting from compression of nerves by medical lines of a type commonly used in surgeries and other medical procedures, while also protecting the medical lines themselves from damage that would interfered with operation for their intended purposes. Further, the nerve injury protection device in accordance with the present invention is compatible with conventional procedures and techniques for protection and restraint of the patient's arms during procedures.

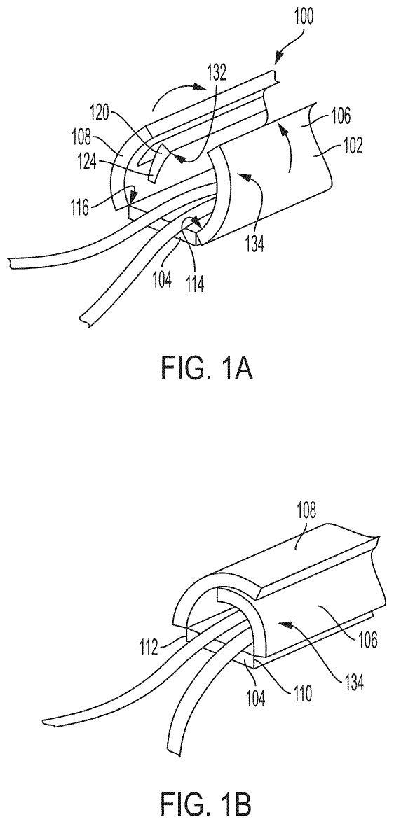

Referring now to FIGS. 1A and 1B, an exemplary embodiment of a nerve injury protection device 100 in accordance with the present invention is shown.

As shown in FIGS. 1A and 1B, the device 100 includes an elongated body 102. The body 102 may be formed as a unitary body that is uniform in cross-section along its length, and thus may be suitable for manufacturing by extrusion. The body 102 is formed of a relatively flexible and readily compressible material, such as a foam material, so as to readily bend and conform any contours of a forearm, etc., and to act as a cushioning material. Further, the material or at least a portion of the material is preferably relatively thick, meaning at least 0.125 or 0.25 inches in thickness or having a thickness that is in the range of at least about 1 to about 5 times as thick as the thickness of the medical lines/elongated members (e.g., including or excluding fittings) with which it is intended to be used, to facilitate the function of a portion of the materials as cushioning, as described below. In other embodiments, the material may have any thickness suitable for decreasing pressure per square unit area to diminish the risk of the nerve or tissue injury from unintended pressure from the medical lines during prolonged procedures in which the medical lines overlie the body. Further, the elongated body may be constructed of a material that is readily cleanable, e.g., disinfectable and/or sterilizable, using cleaning methods of the type typically used for products used in the operating theater, and/or that resists absorption of bodily or other fluids, making it well-suited for use in the operating theater. By way of example, a closed-cell EPDM foam material may be used for this purpose. Alternatively, materials intended to be disposable, and not cleaned and reused, may be used.

The body 102 defines a base 104 provided for abutting the patient's arm/skin, etc. and serving as cushioning to provide a physical buffer between medical lines and the patient's arm/skin/nerves. Further, the body 102 defines a retention flap 106 joined to the base and a closure flap 108 joined to the base 104. In the exemplary embodiment shown in FIG. 1A, the base 104 and flaps 106, 108 are well-defined as discrete portions, and the flaps 106, 108 are joined to the base along opposite longitudinal edges 110, 112 of the base 104. In this exemplary embodiment, the material of the body is continuous across the base 102 and flaps 106, 108, and thus portions 114, 116 of the material of the body 102 may act as living hinges to permit movement of the flaps 106, 108 relative to the base 104 for the reasons discussed herein.

The device 100 further includes a closure for joining the closure flap 108 to the protective flap 106. In the exemplary embodiment shown in FIG. 1A, the closure is provided as a field of adhesive 120 extending longitudinally along the closure flap 108. The adhesive may be a pressure sensitive adhesive, and the field of adhesive 120 may be provided with a removable release sheet 124 that initially covers a surface of the adhesive field and is removable to expose the adhesive for forming an adhesive bond between the closure flap 108 and the retention flap 106 at an appropriate time.

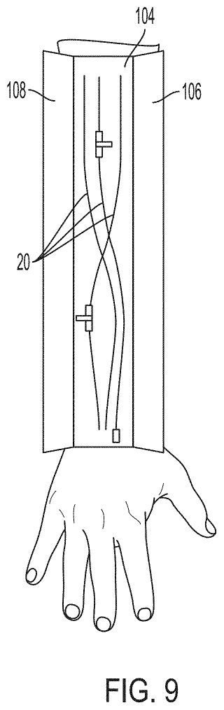

In the exemplary embodiment shown in FIG. 1A, the field of adhesive 120 is provided as a continuous strip, and further, the adhesive field 120 is provided on an inner surface of the closure flap 108. Accordingly, in use, the device 100 may be positioned with its base 104 abutting and extending along/overlying the patient's arm (or other body part, as needed) (see FIG. 9). At this point, the flaps are not secured to one another, and thus they define an open region therebetween for receipt of any medical lines 20 (such as IV or other tubing and associated Luer locks and/or other fittings, as well any cables, leads, and/or wires, such as those used in connection with pulse oximetry and/or blood pressure monitoring apparatuses), as desired, as shown in FIG. 9.

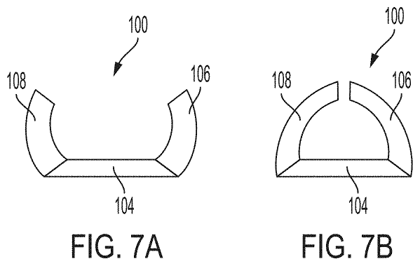

Any desired medical lines may be then be positioned along the patient's arm by placing them in the open region between the open flaps 106, 108, in a manner overlying the base 104, as shown in FIGS. 1A and 1B. In this manner, the base 104 is positioned to and serves as a mechanical buffer and cushioning between the medical lines and the skin of the patient's arm (see FIGS. 7A and 7B). More particularly, the base acts to spread the load of any pressure exerted on the medical lines that would tend to press the medical lines against or into the patient's skin, and by acting as cushioning and/or spreading the load, the base acts to protect the patient from nerve injury due to pressing of the medical lines against the patient's arm. In this exemplary embodiment, the body 102 is manufactured to have a generally flat base 104.

After the medical lines have been positioned between the open flaps 106, 108, and overlying the base 104, as desired, the flaps may be closed, e.g., by joining them to each other. In this embodiment, the retention flap 106 may be folded inwardly to at least partially overlie the base 104 and at least partially enclose/restrain the medical lines, the release sheet 124 may be peeled away to expose the underlying adhesive field 120 on the closure flap 108, and the closure flap 108 may be folded inwardly to cause the closure flap 108 to at least partially enclose/restrain the medical lines and to at least partially overlie the base 104, and also to cause the closure flap 108 to overlie and abut the retention flap 106, and cause the adhesive field 120 disposed on the inner surface 132 of the closure flap to contact and adhere to the outer surface 134 of the retention flap 106, as shown in FIG. 1B. In this manner, a closed region (e.g., 2-3 cm across) is defined as bounded by the base 104 and flaps 106, 108, and medical lines are fully enclosed within the material of the body 102. Accordingly, the material of the body 102 acts to cushion external forces and spread the load of any pressure exerted that would tend to crush, pinch and/or kink the medical lines 20 in a way that would damage them or interfere with their operation as intended. More particularly, the body thereby forms, and particularly in this overlapping-flap embodiment, a protective sleeve strong enough to resist external pressure from surgical arm protectors, arm restrains or other surgical mats or equipment. Further, the low-profile configuration of the device allows for the arm to be secured or restrained in a conventional manner using conventional techniques, e.g., by just wrapping the arm and device together within any sheets, surgical mats/wraps, etc. In particularly, it is noted that the low profile of the device makes it well-suited for use with surgical mats/pads/wraps/supports/cushioning, and thus is compatible with, and not disruptive to, conventional medical procedures or movements.

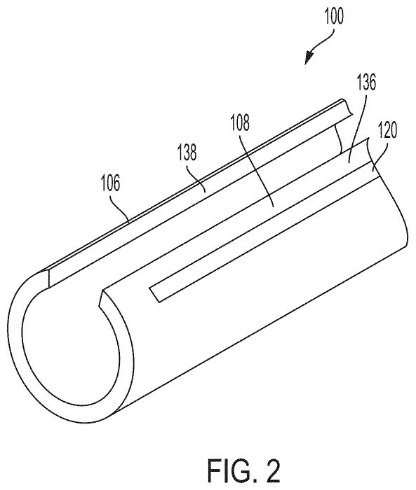

FIG. 2 is a perspective view of an alternative embodiment of the nerve injury protection device 100 of FIGS. 1A and 1B. This embodiment is similar to that of FIGS. 1A and 1B, but in this embodiment, the adhesive field 120 is provided on the outer surface 136 of the closure flap 108, and the flaps are closed by folding the retention flap 106 over the closure flap 108 to secure the adhesive field on the outer surface 136 of the closure flap 108 to the inner surface 138 of the retention flap 106. In this embodiment, the closed flaps again overlap to provide particular resistance to crushing/pinching of the device 100 and any medical lines 20 contained therein. By way of alternative example, in this embodiment, the base 104 and flaps 106, 108 are not well-defined such that the base has well-defined longitudinal edges. Rather, in this embodiment, the material and thickness of the base and flaps are substantially consistent with no clear delineation between the base and flaps. In this exemplary embodiment, the body 102 is manufactured to have a generally curved base 104. For example, the entire body 102 may be formed as a cylindrical/tubular body or a generally-cylindrical/tubular body that has a somewhat flatter side for abutting the patient's skin.

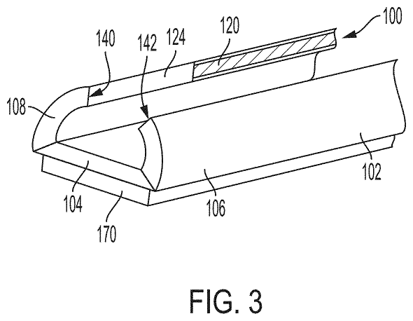

FIG. 3 is a perspective view of a nerve injury protection device 100 in accordance with another alternative embodiment of the present invention. In this embodiment, the base 104 and flaps 106, 108 are again well defined. However, in this embodiment, the adhesive field 120 and release sheet 124 are provided on an and surface 140 of the closure flap the intention of forming and adhesive bond with a complementary edge surface 142 of the retention flap 106. Accordingly, in this embodiment, the flaps 106, 108 will not overlap when joined to one another, but the medical lines will nevertheless be enclosed within the body 102 and thus be protected from damage, while the patient's arm and nerves will be protected by the base 104 of the device 100.

In the exemplary embodiment of FIG. 3, the device 100 further includes a layer of padding 170, such as cotton or another soft material, that is positioned on the base 104 to create a cushioning barrier between the device and the patient's skin. The layer of padding 170 may be supported on the base 104 so as to be readily removable, e.g., via low-tack adhesive or fasteners, if desired.

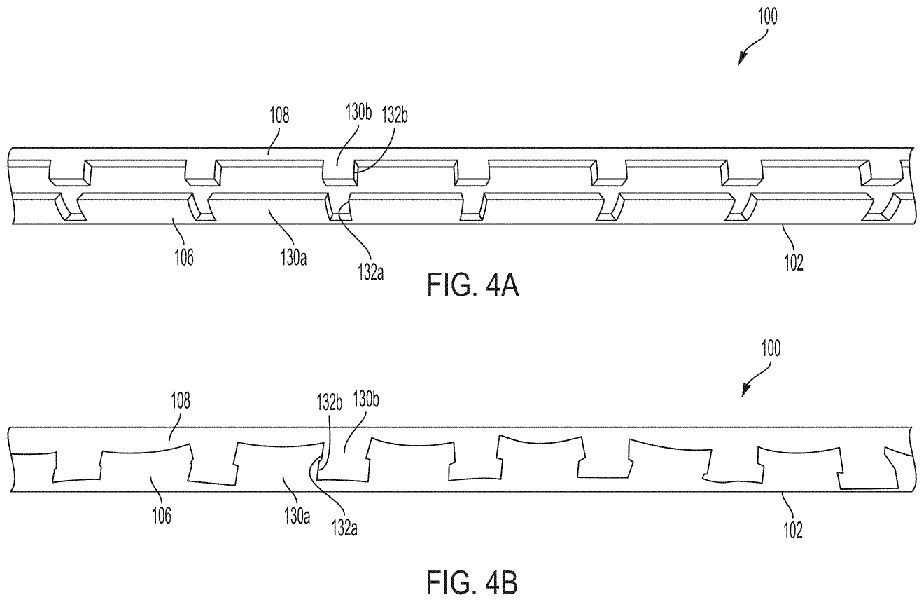

FIGS. 4A and 4B are perspective views of a nerve injury protection device 100 in accordance with yet another alternative embodiment of the present invention. In this embodiment, the base 104 and flaps 106, 108 are not well-defined, e.g., because the device is formed to have a body 102 that is generally circular in transverse cross-section. Unlike the embodiments shown in FIGS. 1-3, which use an adhesive field and release sheet as part of the closure, in this embodiment, edge portions/flaps 106, 108 of the body 102 are specially-configured with matable structures for mechanically joining, e.g., releasably, the edge portions together. More particularly, in the embodiment shown in FIGS. 4A-4B, the body 102 is formed to have one or more edge sections that define a plurality of complementary teeth 130a, 130b separated by spaces that effectively define sockets 132a, 132b therebetween. The teeth 130a and spaces/sockets 132a of one edge/flap 106 are disposed in an interlaced relationship with those of the teeth 130b and spaces/sockets 132b of the opposed edge/flap 108, such that the tooth of one edge is aligned with a corresponding space/socket on the opposite edge. Preferably, each tooth and/or socket is sized and/or shaped to help retain each tooth in its corresponding socket. In the exemplary embodiment of FIGS. 4A and 4B, each space/socket is narrower in width than its associated tooth, such that the tooth (e.g., of a foam material) can be compressed before insertion into the socket, at which point the resiling of the tooth material tends to engage the socket/adjacent teeth and retain the tooth in the socket (as shown in FIG. 4B). When the teeth are disengaged (as shown in FIG. 4A), an open region is defined by the body (between the flaps/edges 106, 108) for receiving wires/lines. Further, when the teeth on one longitudinally extending edge of the body are engaged with complementary teeth on another longitudinally extending edge of the body (as shown in FIG. 4B), the open region is closed and the wires/lines are retained within the closed body, and the teeth are retainer in the closed position, by an interference fit between the complementary teeth/sockets.

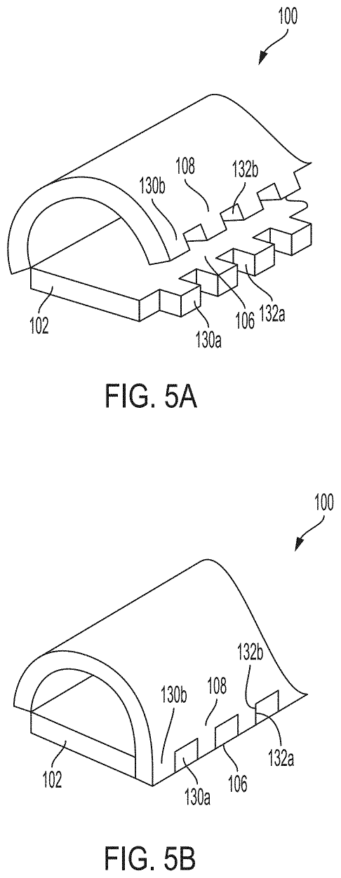

FIGS. 5A and 5B are perspective views of a nerve injury protection device in accordance with yet another alternative embodiment of the present invention. This embodiment is somewhat similar to that shown in FIGS. 4A and 4B, but in this embodiment, the base 104 and flaps 106, 108 are well-defined. Like the embodiment shown in FIGS. 4A and 4B, the flaps/edges 106, 108 are specially-configured with matable structures for mechanically joining, e.g., releasably, the edge portions together, namely, in this embodiment shown in FIGS. 5A-5B, complementary teeth 130a, 130b separated by spaces that effectively define sockets 132a, 132b therebetween. Unlike the embodiment shown in FIGS. 4A-4B, in this embodiment, the base 104 is flat and forms one flap as part of the base, and is joined to the other flap by a living hinge defined by connective material of the body 102. It will be appreciated that in other embodiments, the living hinge may be replaced with additional matable structures for mechanically joining the base and flap portions together, such as complementary teeth and sockets.

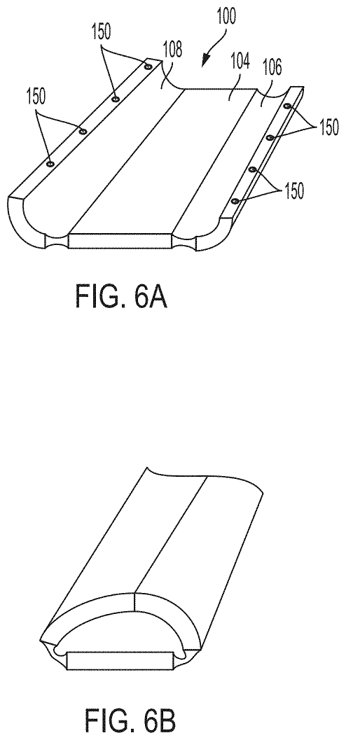

FIGS. 6A and 6B are perspective views of another alternative embodiment of the nerve injury protection device 100. In this embodiment, rather than inclusion of an adhesive field 120, the closure is provided as a plurality of magnets disposed and corresponding longitudinal positions along the edge surfaces 140, 142 of the flaps 106, 108. Accordingly, similar to the embodiment of FIG. 3, the flaps 106, 108 will not overlap. When joined to one another, but the medical lines will nevertheless be enclosed within the body 102 and thus be protected from damage, while the patient's arm and nerves will be protected by the base 104 of the device 100.

FIGS. 7A and 7B are perspective views of yet another alternative embodiment of the nerve injury protection device 100. As shown in FIGS. 7A and 7B, the body may be constructed with living hinges joining the flaps 106, 108 to the base 104, and the living hinges may be structured to tend to assume either an open position or a closed position, and to avoid remaining in a position between the open position and the closed position. Notably, this embodiment does not require adhesive and may include additional material within the hinges (flexible plastic) to provide a snap-open/snap-close feature to these hinges.

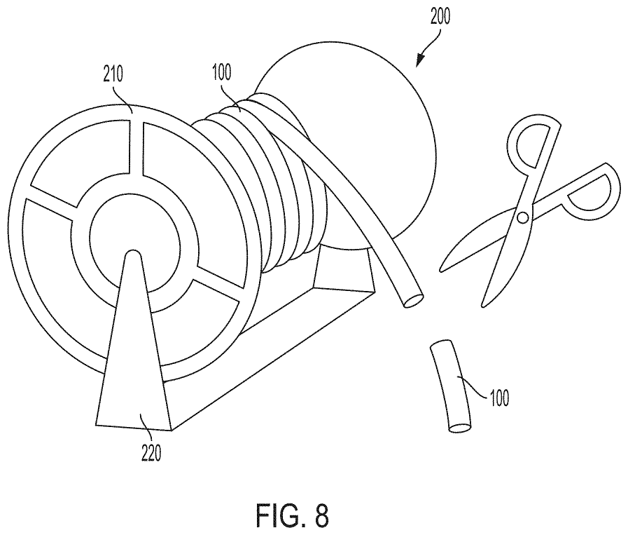

In certain embodiments, the device 100 may be manufactured in lengths suitable for use to extend along the length of the arm without a need to trim the device to length. Rather, the device is manufactured in lengths that are ready-to-use. For example, the device 100 may be manufactured in lengths ranging from about 1 foot in length to about 3 feet in length, or in multiple lengths in 1-2-inch or other-inch increments, for this purpose. Alternatively, the device may be manufactured in lengths much longer than a length intended for use to cushion a patient's arm, e.g., in lengths of 20 feet, 50 feet, hundred feet, or more, and may be packaged in a dispenser device 200 such as the exemplary dispenser shown in FIG. 8. In this exemplary embodiment, the dispenser includes a spool 210 mounted on a support structure 220 and a long length of the device is wound about the spool 210. In use, a desired length of the device may be unwound from the spool and the desired length may be severed from the spool with the use of shears, etc.

It should be noted that in other embodiments, other structures and types of closures may be employed consistent with the present invention. For example, embodiments may use pretensioned members, string, a structural frame, snaps or rigid removable trays that act to retain the body in an open configuration, or urge the body to assume a closed configuration, which closed configuration may or may not require overlapping of flaps or even complete enclosure of a central region by the body. Alternatively, tape, strings, ribbons, sutures or other external fastening elements may alternatively be used to close/partially-close the body, and enclose the medical lines. Alternatively, rather than foam or gel, inflatable body members may be used. Alternatively, integral portions of the flaps themselves (such as complementary S-shaped, S-shaped or hook-type portions) may be included to act as fasteners that may be engaged to join the free ends/edges of the closure flap and the retention flap.

While there have been described herein the principles of the invention, it is to be understood by those skilled in the art that this description is made only by way of example and not as a limitation to the scope of the invention. Accordingly, it is intended by the appended claims, to cover all modifications of the invention which fall within the true spirit and scope of the invention.

What is claimed is:

1. A protective device for protecting a patient's limb from injury resulting from compression against a medical line, said protective device comprising:
a body comprising:
an elongated base;
a retention flap; and
a closure flap; and
a closure supported on said body and operable to join said closure flap to said retention flap.

2. The protective device of claim 1, wherein said base is constructed of a foam material having a thickness greater than 0.125 inches.

3. The protective device of claim 1, wherein said closure is provided as unitary structure integral to said body, said closure and said body being constructed of a single material.

4. The protective device of claim 3, wherein said closure is provided as at least one living hinge joining at least one of said retention and closure flaps to said base, said at least one living hinge being structured to tend to assume at least one of an open position, in which said retention flap and said closure flap are not in an abutting relationship, and a closed position, in which said retention flap and said closure flap are in an abutting relationship.

5. The protective device of claim 1, wherein said closure is a distinct structure attached to, and formed of a different material from said body.

6. The protective device of claim 5, wherein said closure comprises a plurality of magnets.

7. The protective device of claim 5, wherein said closure comprises a field of adhesive.

8. The protective device of claim 7, wherein said field of adhesive is positioned on an inner surface of said closure flap to join said inner surface of said closure flap to an outer surface of said retention flap.

9. The protective device of claim 7, wherein said field of adhesive is positioned on an edge surface of said closure flap to join said edge surface of said closure flap to a respective edge surface of said retention flap.

10. The protective device of claim 1, wherein said base, retention flap, and closure flap are well-defined as discrete portions of said body.

11. The protective device of claim 1, wherein said base is formed as a flat body, and wherein said retention flap and said closure flap are joined to said base.

12. The protective device of claim 1, wherein at least one of said retention flap and said closure flap is joined to said base by a living hinge, said living hinge and said at least one of said retention flap and said closure flap being formed of a single material.

13. The protective device of claim 1, wherein at least one of said retention flap and said closure flap is provided as a portion of said base.

14. The protective device of claim 1, wherein said retention flap and said closure flap are provided with complementary structures matable to mechanically join together said retention flap and said closure flap.

15. The protective device of claim 1, wherein said complementary structures comprise a plurality of teeth defined on one of said retention flap and said closure flap, and a plurality of complementary sockets dimensioned to receive and retain said plurality of teeth on another of said retention flap and said closure flap.

16. The protective device of claim 1, further comprising padding disposed on an external surface of said body to provide a cushion between said protective device and skin of a patient.

17. A protective device for protecting a patient's limb from injury resulting from compression, said protective device comprising:
a body comprising:
an elongated base;
a retention flap joined to said base, said retention flap extending longitudinally of said base; and
a closure flap joined to said base, said retention flap extending longitudinally of said base opposite said retention flap; and
a closure supported on said body and operable to join said closure flap to said retention flap, said closure, said base, said retention flap and said closure flap collectively defining a closed region dimensioned to receive a medical line.

18. The protective device of claim 17, wherein said closure is provided as at least one living hinge integral to said body and joining at least one of said retention and closure flaps to said base, said at least one living hinge being structured to tend to assume at least one of an open position, in which said retention and closure flaps are not in an abutting relationship, and a closed position, in which said retention and closure flaps are in an abutting relationship.

19. The protective device of claim 17, wherein said closure comprises a plurality of magnets.

20. The protective device of claim 17, wherein said closure comprises a field of adhesive.

21. The protective device of claim 17, wherein at least one of said retention flap and said closure flap is provided as a portion of said base.

22. The protective device of claim 17, wherein said retention flap and said closure flap are provided with complementary structures matable to mechanically join together said retention flap and said closure flap.

23. A protective device for protecting a patient's limb from injury resulting from compression, said protective device comprising:
a body comprising:
an elongated base;
a retention flap joined to said base, said retention flap extending longitudinally of said base;
a closure flap joined to said base, said retention flap extending longitudinally of said base opposite said retention flap; and
a closure operable to join said closure flap to said retention flap in a position defining a closed region dimensioned to receive a medical line.

24. The protective device of claim 23, wherein said closure is formed as part of said body.

25. The protective device of claim 23, wherein said closure is a separate body that is not formed as part of said body, but is attachable to said body.

26. The protective device of claim 23, wherein said closure is provided as at least one living hinge integral to said body and joining at least one of said retention flap and said closure flap to said base, said at least one living hinge being structured to tend to assume at least one of an open position, in which said retention flap and said closure flap are not in an abutting relationship, and a closed position, in which said retention flap and said closure flap are in an abutting relationship.

27. The protective device of claim 23, wherein said closure comprises a plurality of magnets.

28. The protective device of claim 23, wherein said closure comprises a field of adhesive.

29. The protective device of claim 23, wherein at least one of said retention flap and said closure flap is provided as a portion of said base.

30. The protective device of claim 23, wherein said retention flap and said closure flap are provided with complementary structures matable to mechanically join together said retention flap and said closure flap.
